(19) United States
(12) Patent Application Publication
(10) Pub. No.: US 2016/0367783 A1
(43) Pub. Date: Dec. 22, 2016

(54) Original, empirical method to create stimulations or activities in the brain, presumably relative to, around some, or in relation with some strongly anchored memories and souvenirs, also meant for therapeutic applications

(71) Applicant: Olivier Dehousse, New York, NY (US)

(72) Inventor: Olivier Dehousse, New York City, NY (US)

(73) Assignee: Olivier Dehousse, New York, NY (US)

(21) Appl. No.: 14/545,790

(22) Filed: Jun. 22, 2015

(51) Int. Cl. A61M 21/00

(52) U.S. Cl. CPC A61M 21/00; A61M 2021/005

(57) ABSTRACT

We aim at creating specific brain stimulation or activities, meant to unify visual brain processing—in creating fast and high definition images in motions (movies), if necessary with specific content, presented in such a way, by its frequency and definition and maybe other parameters to learn, that it is close to what we called the maximum frequency detection, and which the brain will need to process with one or two eyes—and deeply anchored souvenirs and memories, in creating activities or stimulus in specific area of the brain, leading to specific therapeutic applications.

### CROSS-REFERENCE

Provisional patent application (same owner) U.S. 61/465,772 is a related patent. It was introduced as a provisional patent, which foundations use the same principles as here, leading to non-provisional patent (same owner) US 2014/0305209 for the so-called “maximum frequency” detection concept, and to the still pending patent application Ser. No. (14/120,242) exploring further brain processing activities. Furthermore, provisional patent application No. 62/176,304, submitted on Feb. 18, 2015, relying on the field of cognitive dissonance also highlights aspects of our method described here.

### BACKGROUND OF THE INVENTION

Field of Invention

Our invention consists of a method, also meant for therapeutic applications (and in that sense also akin to or capable to enhancing classic Freudian psycho-analysis), which is conceptually directly related to former personal research works, observations and experiments, which we have led and documented, some of which also in the context of previous patents (see cross-reference section). It entails deeper experimentations and explorations of what we have defined in our own patent as the maximum frequency measurement around the so-called wagon wheel effect created with our apparatus (see US 2014/0305209). Our method relies on an apparatus (too simple to be part of this submission) which can present in fast sequences and high definition images in motions (such as in the format of traditional movies), if necessary with specific content, presented in such a way, by its frequency and definition, that it is akin to but below what we have identified as the maximum frequency detection, which can be projected in a specific area of or in the entire the retina, and which the brain can then process with one or two eyes. It is possible to further qualify what these fast and high definition movie-like consists of, in describing the pace of sequence of images as just a few steps below what could be seen as (a sequence of) subliminal images (images which the brain recognize in some ways or shape in an abstract matter, but that appear to fast to be consciously recognized). if necessary in such a way that might require to be specific to each individual, or clusters of individual.

Related Fields, Observations, or Pertaining Observations

We have been able to identify many observations or events, sometimes in reference with our daily life, in relation to the method (and apparatus) suggested and presented here.

For instance, it is public knowledge, and also a definite view supported by many experts, that, at a certain point of the evolution, the homo sapiens might have started to find more efficient to hunt larger animals by scaring them with the goal to try to create suffocating symptoms in them, and as such enabling them to be captured. Such theory is also at the cross of our analysis, which is to say, that, based on what we have experienced ourselves, the human body, and for that matter, the body of an average homo sapiens, to whom we think our observation might also be relevant, and which constitution, for someone who is physically well trained and exercises frequently, probably translates into what our average ancestors homo sapiensbeing smaller and more muscular, could do, seems to be optimized for running (or also called jogging type) activities, under sustained and continuous effort, distance of about 10 km (or about 6.2 miles), regularly, f.i. daily if necessary, and do such over a period of about 50 minutes to an hour. It is important to note also that such analysis which we place here in the context of the human evolution, corresponds more or less also to what is known and used as the period of maximum concentration (more or less 50 minutes) for young adults (teenagers) at an age where the human body is also fully formed. It is therefore possible that these 50 to 60 minutes is the result of a long sequence of optimization in the human body, and correspond to the time required to hunt, and win over large animals by suffocations, and possibly reconnect with part of the community left behind and consequently make the entire community benefit from such gain. From there, we could potentially also assume that such adaptations considered in the context of the evolution might have found an optimum, allowing to further evolving on other characteristics of our brain, such as a more abstract intelligence, other learning capabilities, and more.

In this context, we also wonder if a large number of cases of attention deficit disorders could not come from the fact that in many daily life experience, the brain is sub-optimized in a way that it is far from using these (more or less) 50 minutes of maximum concentration periods.

Other phenomena relative to our methods are already present in early form of pedagogy, such as the one already used by Aristotle, which consisted of walking and learning at the same time.

Other adages, which are presumably derived from large number of experiments and observations, such as “travelling maintain a young (and sharp) mind”, could also be seen as in alignment with what we are trying to describe here, and which might also validate further that fact that our method could indeed be used for therapeutic applications.

It further appears that even today, as a potential consequence of our long evolution, humans are getting rewards when analyzing imaging in motions, which is the case in most sports, watched or practices . This aspect and observation have already been submitted as part of our former patent application.

Conversely, when a sequence of images starts to appear as too slow, or with too little definition, it would appear such as creating the inverse effect and create relaxation and sleepiness feelings.

Furthermore, and as already submitted with an earlier patent submission. there are multiple reasons leading to think that the fascination for fire, which is found more or less within a majority of human beings, is once again illustrating that this phenomena, which supports our method here also, could be considered to be at the birth of the human being intelligence development. Indeed, human beings of all kinds during the evolutionary process might have encountered fires created naturally around them (we assume here indeed that fires were at the origin created naturally), but it had to wait for superior human intelligence to be developed, or f.i. the right genetic mutation for instance, which once it has occurs needs to be passed along, as per our theory developed here, to become the source of a fascination and as consequence to become the object of domination and of such attempts, and which would have taken place around the same time as the aptitude (and reward) for analyzing images in fact motions, also supporting evidence for our method described here. If so, we could indeed not be too far at all from—in fact right there on board with—the Prometheus myth, which some see, as the birth for evolution (and consequently intelligence). And therefore, we have many reasons to believe that such point in time along the evolution, might constitutes what we could look at as the birth of a nascent abstract “intelligence”, not only from a memorization stand points, but also from other stand points, such as communications, orientations in new environment, notions of times, and period, anticipations, and more.

On a separate note, our method also relies further on some aspects of the already well established field of knowledge called cognitive dissonance, and in specific also to another patent that we have submitted.

### BRIEF DESCRIPTION OF THE DRAWINGS

Not applicable here, due to the simplicity of the apparatus on which our method relies

### DETAILED DESCRIPTION OF THE METHOD

Our original method relies on the above observations, (using empirical theories, or effective field theories), and could also be seen is the logical and necessary conclusion of the sequence of evidences presented here above.

Our method is also meant to enhance what is traditionally seen as Freudian (and subsequent disciples) psycho-analysis therapy, and therefore is meant to potentially present some therapeutic values, in the field of psycho-analysis and or cognitive sciences.

In short, our method consists of creating specific brain stimulation or activities, meant to unify visual brain processing—akin to what we described here above in creating fast and high definition images in motions (movies), if necessary with specific content, presented in such a way, by its frequency and definition and maybe other parameters to learn, that it is close to what we called the maximum frequency detection, and which the brain will need to process with one or two eyes - and souvenirs or memories in creating center of activities or stimulus, for therapeutic reasons.

Therefore, it is likely that the content of such movies—see above—presented should be in close relation, or intertwined with the area of memory or souvenir, origin of the neurosis, which is of therapeutic interest, in the usual way defined by the Freudian theory.

Potentially, other mental deficiencies or mental illness, such as (but not limited to) attention deficits disorders, nascent Alzheimer symptoms, or more, could be seen as field of application for our method, per the definition from above, with or without specific content, and with or without other attributes.

### SUMMARY OF THE INVENTION

In accordance with our present invention, a method, meant for therapeutic applications (and ins such sense also akin to or enhancing classic Freudian psycho-analysis), which is conceptually directly related to former work and experiment which we have led and documented, also in the context of a previous patent. It consists of further experimentations and exploration of what we have defined in our own patent as the maximum frequency measurement around the so-called wagon wheel effect created with our apparatus. Our method relies on an apparatus (too simple to be part of this submission) which can create fast and high definition images in motions (movies), if necessary with specific content—content which should be in close relation with the area of memory or souvenirs, which is of therapeutic interest, in the usual way defined by the Freudian theory—, presented in such a way, by its frequency and definition, that it is akin to what we called the maximum frequency detection, and which the brain can process with one or two eyes. It is possible to further qualify what these fast and high definition movies consists of, in describing them as just a few steps below what could be seen as (a sequence of) subliminal images (images which the brain recognize but that appear to fast to be consciously recognizable), considering that such quantitative and qualitative attribute might be specific to each individual.

Our method could be seen as an enhancement of what is usually understood under Freudian psycho-analysis and associated therapeutics, and it could also pertain to other type of mental illness or symptoms, such as (but not limited to) attention deficit disorders, early Alzheimer symptoms, and others brain degenerative diseases.

1. A method to create specific brain stimulation or activities, meant to unify visual brain processing
in creating fast and high definition images in motions (movies), if necessary with specific content, presented in such a way, by its frequency and definition and maybe other parameters to learn, that it is close to what we called the maximum frequency detection, and which the brain will need to process with one or two eyes - and deeply anchored souvenirs and memories, in creating activities or stimulus in specific area of the brain, leading to therapeutic applications.
